(19) United States
(12) Patent Application Publication
(10) Pub. No.: US 2014/0209594 A1
(43) Pub. Date: Jul. 31, 2014

(54) Far Infrared Heat Emitting Therapeutic Device

(71) Applicant: Robert Besner, Boca Raton, FL (US)

(72) Inventor: Robert Besner, Boca Raton, FL (US)

(21) Appl. No.: 14/230,358

(22) Filed: Mar. 31, 2014

Related U.S. Application Data
(63) Continuation-in-part of application No. 12/485,807, filed on Jun. 16, 2009, now abandoned.
(60) Provisional application No. 61/061,813, filed on Jun. 16, 2008.

(51) Int. Cl. A61F 7/00

(52) U.S. Cl. CPC A61F 7/007
USPC 219/217; 607/100

(57) ABSTRACT

A far infrared heat emitting device includes a pad for positioning over or around a body part of a user such as a waist, leg, arm, or back. The user may be a human or an animal such as a pet. The device can include straps to form a strap sized to fit around the waist of a human user. The strap includes a pad, a plurality of far infrared heat conducting elements, a charging inlet, a heating device, and a fastener for securing the strap to the human user. The device may be secured around the waist of the human user to provide relief from lower back pain. Alternatively, the device may be used in or on furniture. The device may produce an aura of negative ions. The device may minimize the presence of at least some electromagnetic frequencies.

### CROSS-REFERENCE TO RELATED APPLICATIONS

This application is a continuation-in-part application of U.S. nonprovisional patent application Ser. No. 12/485,807 filed on Jun. 16, 2009, which is a nonprovisional application of and claims priority from U.S. provisional patent application Ser. No. 61/061,813 filed on Jun. 16, 2008. The foregoing applications are incorporated in their entirety herein by reference.

### FIELD OF THE INVENTION

The invention relates to devices and methods for providing healing or curative effects for health conditions as well as pain relief and relaxation. More particularly, the invention relates to devices and methods that incorporate a far infrared heating system that is useful for relaxing muscles, relieving pain, and increasing blood circulation.

### BACKGROUND

One of the most common medical complaints is lower back pain. Lower back pain is associated with inflammation of the nerves, muscles, bones, connective tissue, or joints and may result from a number of different causes. Among the many different treatments for lower back pain, heat treatment has been shown to be both effective and easy to administer.

Conventional heat emitting therapeutic devices have utilized heated water in the form of hot water bags and bottles, oil containing bags or bladders that can be heated, and electric blankets. These conventional heating devices heat the body's surface through heat transfer by heat conduction between the heating device and the skin. Often, the thermal radiation transferred as heat between two surfaces may not penetrate deeply enough into or beneath the skin to provide any useful curative or pain relief effects. In addition, because of the nature of human skin protection for our bodies, conventional heating emitting therapeutic devices may only be used for a short period of time before burning of the skin occurs.

Infrared heat solves some of the challenges of discussed above without creating new challenges, contraindications, or physiological limitations. And this specific form of thermal energy is now documented as beneficial not only for lower back pain; it has been successfully documented to assist users in detoxification (i.e. removing harmful toxins from the body). FIR heat has been shown in clinical studies to treat the multiple risk factors of cardiovascular disease such as normalizing blood pressure, lowering cholesterol, reducing pain, stress, and fatigue, and weight loss. Addressing these risk factors may have a synergistic effect in addressing congestive heart failure, hypertension, obesity, and Type-2 diabetes. What is needed is a solution that provides infrared heat that is efficiently and substantially evenly distributed to a user.

### SUMMARY

The therapeutic device of the present invention advantageously provides a heating solution that provides infrared heat that is efficiently and substantially evenly distributed to a user. An ancillary but equally important benefit in providing healing or curative effects by the invention is the circumstance that the stones or minerals used may create an aura of negative ions when charged with infrared heat. Negative ions are odorless, tasteless, and invisible molecules that are absorbed in abundance in certain environments and enter the bloodstream. By providing an aura of negative ions, the present invention advantageously facilitates biochemical reactions that increase levels of chemicals, such as serotonin, to help reduce the effects of depression, stress, and lethargy. The reaction of the heat-conducting stones or minerals acts as a primer for the creation of negative ions that are emitted from the device.

One of the hidden toxins of modern life is what is commonly referred to as “electrosmog”, the broad area of devices, including electronic devices other than the device of the present invention, which create electromagnetic radiation. The countless wireless devices coupled with the over-abundance of electrical appliances team up to create a harmful, invisible environment that is only recently being addressed. Through proper shielding included in the subject device, only a minimal level of EMF exists in this invention, making this invention fundamentally different than prior inventions and unique in the marketplace

The invention relates to far infrared (FIR) heat emitting devices that generate heat in the form of FIR radiation that is emitted and transferred both by radiation and conduction to a human's skin surface for conduction into and penetration beneath the skin. Due to the wavelength of FIR thermal waves, FIR can penetrate more deeply into and beneath the skin surface to provide curative, pain relieving effects for tissue beneath the surface of the skin.

The FIR heat emitting device can be a therapeutic device that emits FIR heat that can be absorbed by the human body, and particularly, through human skin and sub-dermal tissues, through FIR heat radiation and conduction. The therapeutic device can be a pad that features heat emitting, heat conducting, and heat radiating elements. The FIR heat emitting device can include a frequency generating component connected to a power source and integrated with a component that conducts the heat created by the thermal frequency, emitting the infrared heat through the device and/or pad.

Far infrared heat conducting and radiating elements (e.g., jade discs) installed on an exterior surface of the pad can be exposed to some of the radiating far infrared heat emitted by the resonating element. As the FIR heat is emitted, the thermal frequency resonates with the frequencies of the human body's cellular structure, penetrating the user's skin and tissues, and creating a biothermal reaction of internal warmth. As this thermal heat warms the user's body, it increases the user's core temperature improving blood circulation, providing pain relief and relaxation, and restoring physiological balance.

The heat-conducting elements can be a plurality of stone or mineral members such as, for example, jade. The heating elements can be arranged in a regular pattern on an inner or outer surface of the pad. The stone or mineral members can be whole, crushed, granulized, or miniaturized and impregnated. The pad can further include a charger to permit connection to a power source.

In some embodiments, the pad may include one or more fasteners to form a strap that can be attached around a body part such as, for example, a waist, arm, leg, or neck. The fasteners can be straps that can include a buckle, button, or hook-and-loop fastening means.

The FIR heat emitting strap can conveniently be worn around the waist of a human subject to relieve lower back pain. In preferred embodiments, the strap is shaped somewhat like a conventional weight lifting belt for the purpose of securing around a user's waist and is flexible. Far infrared heat can be delivered to a user via jade discs arranged in a regular pattern on or beneath a front surface of the device such that the jade discs are positioned in contact or near contact with the user when the device is being worn. A device extension, or an optional elastic component of the device, can be used to secure the device to a chair.

According to an embodiment of the present invention, a therapeutic device is provided for positioning over or around a body part of a user. The device includes a pad including a front surface and a rear surface, a plurality of far infrared heat conducting elements, a heating device, a power source, and plurality of stones or minerals, which may be free-floating, integrated in the pad by installation within a plurality of cells that are closed to prevent the stone from falling out or becoming dislodged.

In another aspect, the stone may be one or more stones and may further include one or more of jade, granite, tourmaline, or a combination thereof. The one or more stones may be whole, crushed, granulized, miniaturized, and/or impregnated.

In another aspect, the one or more stones may be a primer for creation of negative ions to be emitted.

In another aspect, the pad includes a strap to substantially secure the pad around the body part of the user.

In another aspect, the strap is integrated with the pad as a single piece.

In another aspect, the strap is elastic and extendable for securing the pad around a waist of the user having a waist size that is 36 inches or greater.

In another aspect, the device is sized so as to be capable of attaching around a chair. The device may optionally include a strap to substantially secure the pad to the chair. The chair may include a chair, chair seat, similar seating. The device may optionally be secured internally within the chair.

In another aspect, the stone is at least partially crushed and integrated into the pad to create a negative ion environment. Optionally, the stone may be crushed, granulized, miniaturized, and/or impregnated.

In another aspect, the power source includes a power cord for connection to an outlet of an electrical system, a battery, or a combination thereof.

In another aspect, the front surface of the pad includes a pouch. The pouch may include a lining. The lining can include front and rear interior surfaces.

In another aspect, the pad may include individual cells sewn into the lining. Each of the plurality of far infrared heat conducting elements may be positioned within one of the individual cells sewn into the lining.

In another aspect, the lining may include a mesh material with a plurality of apertures to permit unblocked emission of far infrared heat from the plurality of far infrared heat conducting elements.

In another aspect, the heating device includes an infrared frequency generating device.

In another aspect, the infrared frequency generating device may include a carbon-based material installed inside a pouch of the pad and constructed in a shape that provides close proximity to at least a majority of the plurality of far infrared heat conducting elements.

In another aspect, an aura of negative ions is emitted by operating activated carbon fiber at thermal frequencies.

In another aspect, the pad includes a bioceramic infused FIR fabric.

In another aspect, the pad includes a distribution material to substantially uniformly distribute heat further including jade, granite, tourmaline, or a combination thereof.

In another aspect, the plurality of far infrared heat conducting elements is connected to a mesh material, the front surface, or a combination thereof via a connecting means.

In another aspect, the heating device includes a carbon fiber matrix.

According to an embodiment of the present invention, a therapeutic device is provided for integrating into furniture. The device may include a pad adjacent to a surface of the furniture. The device may also include a plurality of far infrared heat conducting elements. Additionally, the device may include a stone affixed to at least one surface of the pad via a fastener to distribute heat. The device may also include a heating device with a carbon fiber matrix and a power source.

Unless otherwise defined, all technical terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents and other references mentioned herein are incorporated by reference in their entirety. In the case of conflict, the present specification, including definitions will control.

### BRIEF DESCRIPTION OF THE DRAWINGS

FIG. 1 is a front view of a pad embodiment of the invention.

FIG. 2 is a rear view of the pad embodiment of FIG. 2.

FIG. 3 is a front view of a strap embodiment of the invention.

FIG. 4 is a rear view of the strap embodiment of FIG. 3.

FIG. 5A is a top view of one side of a short strap extension capable of being connected to and used with the strap embodiment of FIG. 3.

FIG. 5B is a top view of a reverse side of the short strap extension of FIG. 5A.

FIG. 6A is a top view of one side of a long strap extension capable of being connected to and used with the strap embodiment of FIG. 3.

FIG. 6B is a top view of a reverse side of the long strap extension of FIG. 6A.

FIG. 7A is a front perspective view of the strap embodiment of FIG. 3 connected to a long strap extension and attached around a chair.

FIG. 7B is a rear perspective view of the strap embodiment of FIG. 3 connected to the long strap extension as shown and attached around a chair.

### DETAILED DESCRIPTION

The present invention is best understood by reference to the detailed drawings and description set forth herein. Embodiments of the invention are discussed below with reference to the drawings; however, those skilled in the art will readily appreciate that the detailed description given herein with respect to these figures is for explanatory purposes as the invention extends beyond these limited embodiments. For example, in light of the teachings of the present invention, those skilled in the art will recognize a multiplicity of alternate and suitable approaches, depending upon the needs of the particular application, to implement the functionality of any given detail described herein beyond the particular implementation choices in the following embodiments described and shown. That is, numerous modifications and variations of the invention may exist that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

The present invention should not be limited to the particular methodology, compounds, materials, manufacturing techniques, uses, and applications, described herein, as these may vary. The terminology used herein is used for the purpose of describing particular embodiments only, and is not intended to limit the scope of the present invention. As used herein and in the appended claims, the singular forms “a,” “an,” and “the” include the plural reference unless the context clearly dictates otherwise. Thus, for example, a reference to “an element” is a reference to one or more elements and includes equivalents thereof known to those skilled in the art. Similarly, for another example, a reference to “a step” or “a means” may be a reference to one or more steps or means and may include sub-steps and subservient means.

All conjunctions used herein are to be understood in the most inclusive sense possible. Thus, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should be read as “and/or” unless expressly stated otherwise. Structures described herein are to be understood also to refer to functional equivalents of such structures. Language that may be construed to express approximation should be so understood unless the context clearly dictates otherwise.

Unless otherwise defined, all terms (including technical and scientific terms) are to be given their ordinary and customary meaning to a person of ordinary skill in the art, and are not to be limited to a special or customized meaning unless expressly so defined herein.

Terms and phrases used in this application, and variations thereof, especially in the appended claims, unless otherwise expressly stated, should be construed as open ended as opposed to limiting. As examples of the foregoing, the term “including” should be read to mean “including, without limitation,” “including but not limited to,” or the like; the term “having” should be interpreted as “having at least”; the term “includes” should be interpreted as “includes but is not limited to”; the term “example” is used to provide exemplary instances of the item in discussion, not an exhaustive or limiting list thereof; and use of terms like “preferably,” “preferred,” “desired,” “desirable,” or “exemplary” and words of similar meaning should not be understood as implying that certain features are critical, essential, or even important to the structure or function of the invention, but instead as merely intended to highlight alternative or additional features that may or may not be utilized in a particular embodiment of the invention.

Those skilled in the art will also understand that if a specific number of an introduced claim recitation is intended, such an intent will be explicitly recited in the claim, and in the absence of such recitation no such intent is present. For example, as an aid to understanding, the appended claims may contain usage of the introductory phrases “at least one” and “one or more” to introduce claim recitations; however, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C” is used, in general, such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.).

All numbers expressing dimensions, quantities of ingredients, reaction conditions, and so forth used in the specification are to be understood as being modified in all instances by the term “about” unless expressly stated otherwise. Accordingly, unless indicated to the contrary, the numerical parameters set forth herein are approximations that may vary depending upon the desired properties sought to be obtained.

The soft, flexible invention provides a therapeutic device that may be a far infrared (FIR) heat emitting device, which can be worn or placed over, under, or around a body part of a user such as, for example, a waist, an arm, a leg, a neck, a torso, a hand, a foot, or a back of a human subject. In another embodiment, the user of the invention described herein can be an animal such as a domestic animal, a pet, a farm animal, or a work animal, e.g., a dog, a cat, a horse, a cow, a pig, or any other animal on which a FIR heat emitting device may be used.

In one exemplary embodiment, the FIR heat emitting device 10 can include a pad 12 that is laid or placed over and in contact with or wrapped around a body part of a user. The pad may have a front surface and a rear surface. In another exemplary embodiment, the pad 12 can be equipped with one or more fasteners to form a FIR heat emitting device that is a belt or a strap 100 Skilled artisans will appreciate belts and straps to be related, and are used throughout this disclosure with continuing reference to one another. The strap 100 can be attached around a body part of the user. The FIR heat emitting device can further include a plurality of far infrared heat conducting elements 14. In at least one embodiment, the strap and the pad may be included as one single piece.

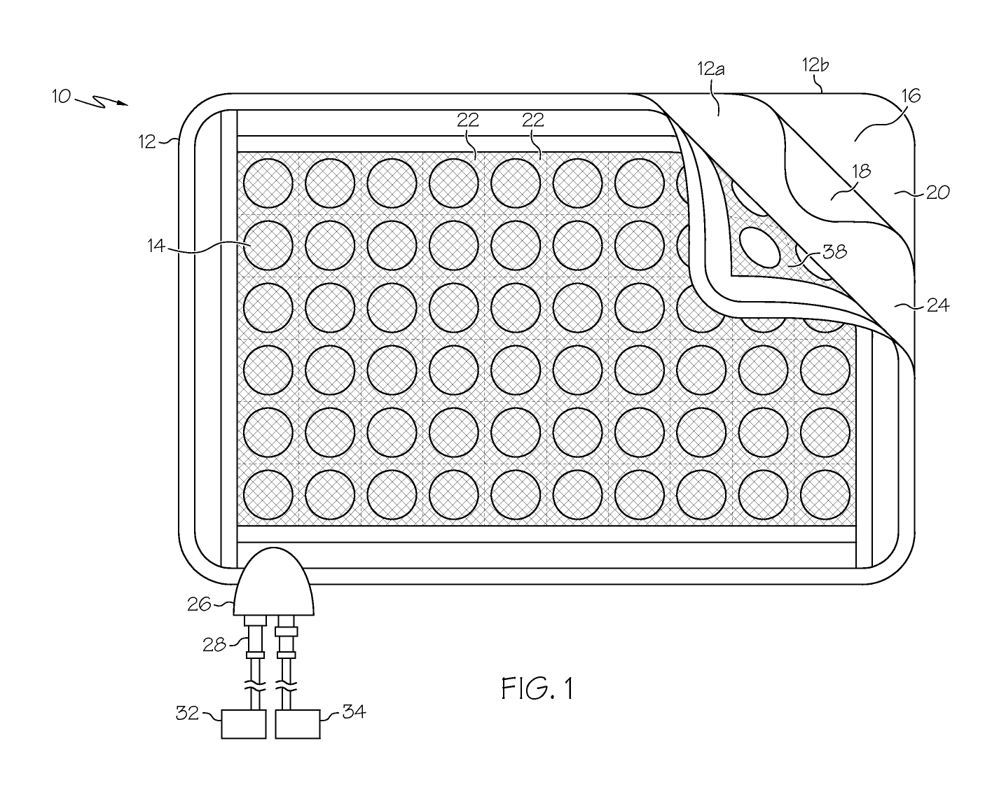

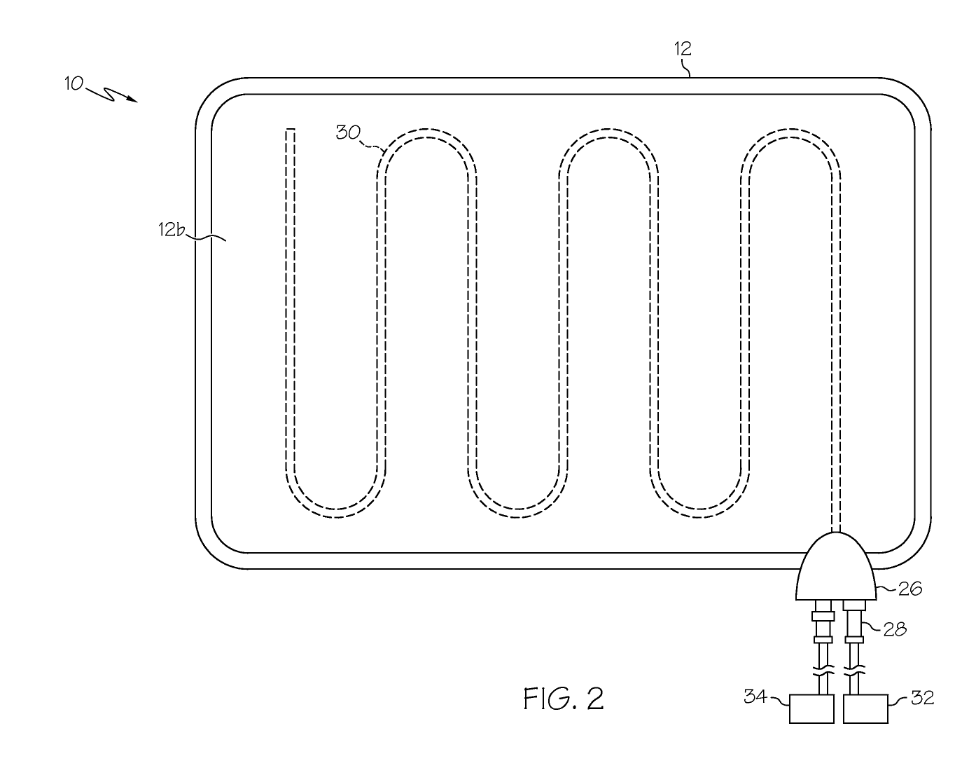

In an exemplary embodiment shown in FIGS. 1 and 2, the pad 12 can be generally flat having a front sheet of material 12a and a rear sheet of material 12b connected together around at least an outside edge of each sheet of material. The front sheet of material 12a and the rear sheet of material 12b may be attached together by stitching. The front and rear sheets of material 12a and 12b may define an empty space between them so as to form a pouch 16. The pouch 16 can be defined by an interior surface 18 of the front sheet of material 12a and an interior surface 20 of the rear sheet of material 12b. The interior surfaces 18 and/or 20 may include a lining, which may include a layer or layers of insulated material.

The device 10 can include a heating device 30. The heating device 30 can be a frequency generating device 30. In at least one embodiment, the heating device 30 can be an infrared frequency generating device. The heating device 30 may include a wiring matrix, which may be a carbon fiber wiring matrix, capable of generating one or more infrared frequency. In at least one embodiment, the wiring matrix is inseparable from the heating device. The frequency generating device 30 can be installed in or on the pad 12 and can be capable of generating a frequency that causes the plurality of FIR heat conducting elements 14 to resonate or vibrate. This frequency may be defined as one or more FIR frequencies. As the plurality of FIR heat conducting elements 14 vibrate, the elements generate heat in the form of far infrared heat radiation that can be emitted by the elements onto the skin of the user.

In an exemplary embodiment, the frequency generating device can feature a carbon-based, polygraphite element 30 as shown in FIG. 2. In another embodiment, the frequency generating device 30 can be constructed from any other suitable carbon-based material, ceramic, or composite stone material. Throughout this disclosure, the term “stone” shall be understood to include minerals and other similar compositions. The frequency generating device can be curvy or winding in shape so as to provide close proximity to the at least the majority of the FIR heat conducting elements. The frequency generating device 30 can receive electrical power from its connection to a power transfer junction box 26 that can be connected to a power cord 28. The power cord 28 can be connected to a power source 32 such as, for example, an outlet connected to an electrical system or electric grid. The power source can be direct current or alternating current and may be provided by a wall outlet, connection to an electrical power grid, and/or a battery. The frequency generating device 30 can be installed inside the pouch 16 so as to be hidden from view when the front and rear sheets of material 12a and 12b are stitched together.

As the polygraphite element 30 resonates while connected to a power source, the resonance produces far infrared radiation. The FIR removes substantially any electromagnetic radiation from the environment. The polygraphite element may also produce other electromagnetic radiation from the infrared spectrum. The far infrared radiation produced by the resonating polygraphite element 30 is emitted as radiation through the front sheet of material 12a of the pad 12 to penetrate the user's skin. In addition, the far infrared radiation emitted by the polygraphite element 30 can cause the FIR heat conducting elements 14 to resonate and to emit additional far infrared radiation. The far infrared radiation produced by the FIR heat conducting elements 14 can penetrate the user's skin via conduction, or direct contact between the user's skin and FIR heat conducting elements.

The device 10 can further include a remote control 34 that may feature controls such as, for example, temperature control, a timer (e.g., a digital timer), a sleep timer with automatic power shut-off, and a power switch to turn the device off and on. The temperature control may include displays connected to sensors positioned within the pad to monitor and alert the user as the device 10 reaches the desired temperature (e.g., a temperature selected by the user). The remote controller can include a display screen, which may include LED or LCD modalities. The display screen of the remote control 34 may include a backlight (e.g., a xenon backlight). The remote control 34 may be connected to the device 10 via a wired or a wireless connection. The remote control 34 may be either digital or analog. The remote control 34 may include an auto-safety shutoff switch.

A mesh material 38 can be attached to an exterior surface 24 of the front sheet of material 12a. The mesh material 38 can be sewn or stitched to attach it to the front sheet of material 12a, for example, via a lining. In an exemplary embodiment, the mesh material 38 may be stitched to the exterior surface 24 of the front sheet of material 12a to form a plurality of individual cells 22. Each of the plurality of cells 22 may contain an individual FIR heat conducting element 14. The cells 22 may be stitched or sewn substantially or completely closed to prevent the plurality of FIR heat conducting elements 14 from falling out or otherwise moving in position or becoming dislodged. In another embodiment, each cell may contain more than one FIR heat conducting element 14. The mesh material 38 can include a plurality of apertures to permit unblocked emission of far infrared heat from the FIR heat conducting elements 14 and radiation and conduction of that far infrared heat to the user's body part that is covered by the pad 12 of the device 10. In an alternate embodiment, a solid sheet of material may be attached to the front sheet of material 12a rather than mesh material.

The material on the exterior of the treatment area of the pad may accept and/or hold stones or minerals to assist in the even distribution of the heat. This material may be a distribution material. Additionally, in an embodiment utilizing contemporary nanotechnology, the material may be a bioceramic infused FIR fabric. Such a bioceramic infused FIR fabric is a functional product composed of woven or non-woven cloth integrated with mineral composites that actively react to the far infrared heat radiating from virtually any living being.

In an exemplary embodiment, the plurality of FIR heat conducting elements 14 can be arranged and fixed in a regular pattern in the cells 22 of the mesh material 38. The FIR heat conducting elements may also be arranged and fixed in an irregular pattern in the cells 22 of the material 38. In another embodiment, the plurality of FIR heat conducting elements 14 can be fixed in a regular or irregular pattern within the pouch 16 of the pad 12. The FIR heat conducting elements 14 can be sewn into the front sheet of material 12a so that each element is fixed in position. For example, the FIR heat conducting element may be free-floating and may be sewn into individual cells provided by a lining of the pouch. The FIR heat conducting elements 14 may also be connected to the mesh material 38 or to the exterior surface 24 of the front sheet of material 12a by other connecting means such as, for example, snap fasteners, clips, adhesives, plastic ties, sewing or stitching using thread or yarn, hook-and-loop, or any other suitable connecting means. The connecting means used to attach the FIR heat conducting elements 14 to the pad 12 may be heat resistant and fire resistant as are the other materials used in the construction of the device 10. The materials used in the construction of the device 10 can also be sealed for water resistance.

In another embodiment, the front sheet of material 12a of the pad 12 can include a second pouch formed from an additional sheet of material attached to either the exterior surface 24 or interior surface 18 of the front sheet of material. The plurality of FIR heat conducting elements 14 can be attached to either the exterior surface 24 or to the interior surface 18 of the front sheet of material 12a. In another embodiment, the FIR heat conducting elements 14 can be attached to both the interior and the exterior surfaces 18, 24 of the front sheet of material 12a of the pad 12.

In one alternate embodiment, the rear sheet of material 12b of the pad 12 can include a charging inlet for connection to a power source. Where the power source is an electrical system, the device 10 may be connectable to the power source via an electrical cord that can be plugged into an outlet. The charging inlet may be enclosed within a pocket that can be hidden by closing a zipper. In another embodiment, the rear sheet of material may include a pocket that can contain a power source that is a battery.

The power source may have parameters of such nature, consistency, and reliability so as to minimize electrical arcing and support the required thermostats in the device. The thermostats may be integrated into the device to prevent burning, exceeding indefinable maximum temperature (for example, 140° F.), or otherwise affect the operation of the device. Additionally, the power source may include support for the remote control device to control temperature, timer, power, related displays, and other aspects of the device.

In one embodiment, the front sheet of material 12a can be two-ply in that it is constructed from two attached sheets of material that may enclose a pouch. Similarly, the rear sheet of material 12b can be two-ply in that it may also be constructed from two attached sheets of material that may enclose a pouch. In another embodiment, the front sheet of material and the rear sheet of material may each be two-ply and when sewn together may enclose a central pouch.

The pad 12 (and the front and rear sheets of material 12a and 12b) may be constructed from any suitable material, e.g., a synthetic or natural material such as vinyl, nylon, polyester, leather, suede, or cotton. The pad 12 can be flexible and may be elastic. The pad 12 may also be constructed from a material that is antimicrobial and antifungal. In another embodiment, the pad 12 may be treated with a composition that imparts antimicrobial and antifungal characteristics to the pad. The pad 12 can be sized to fit over a body part of a human user such as an adult or child. In another embodiment, the pad can be sized and shaped to fit over or around a body part of an animal.

The FIR heat conducting elements 14 can be a stone or mineral material. For example, the FIR heating elements can be jade, germanium, charcoal, rock, porcelain, ceramic, or tourmaline ceramic. The FIR heat conducting elements 14 may be constructed in the shape of discs (circular, oval, or oblong), tiles (square, triangular, rectangular, or rhomboidal), or cubes or the FIR heat conducting elements may be cylindrical, conical, polygonal, or prismoidal in shape. The surface of each FIR heat conducting element can be smooth, convex, concave, or may include a regular or irregular surface texture. Additionally, the heat conducting element, which may include the stones, may be whole, crushed, granulized, miniature, and/or impregnated. The FIR heat conducting elements 14 included with each device 10 may be of the same material, shape, and texture or may vary in material, shape, and/or texture. The FIR heat conducting elements 14 can be arranged in a regular or geometrical pattern or in an irregular pattern. In an exemplary embodiment, the FIR heat conducting elements 14 are jade discs.

In another embodiment, the FIR heating elements 14 can be a man-made, artificial heat conducting or emitting material that may be similar in consistency to stone.

According to an embodiment of the present invention, carbon fiber may be used as the conductive vehicle for emitting far infrared frequencies. Although stones, for example jade, are included to transmit the far infrared radiation, various embodiments of the present invention do not required such stones for conductivity in the emission of far infrared frequencies. In one example, a carbon fiber matrix may be energized to generate infrared frequencies. The stones of the present invention may be used to aid and assist in even distribution of the thermal heat, but may not be required to create or emit the infrared heat. Because of this novel configuration of the present invention, thermal frequencies may be generated in the device itself, thus heat may be provided to a user without requiring a hard connection to a far infrared radiator.

The stones, for example, jade discs, may create an aura of negative ions. The negative ions may be odorless, tasteless, and invisible molecules that are absorbed in abundance in certain environments and enter the bloodstream. By providing the aura of negative ions, the present invention advantageously facilitates biochemical reactions that increase levels of chemicals, such as serotonin, to help reduce the effects of depression, stress, and lethargy. The stones may act as a primer for the creation of negative ions that can be emitted from the device. For example, without limitation, stones including jade, granite, tourmaline, or combination thereof may be used as a primer for the creation of negative ions.

The invention 10 may feature a storage bag or a carrying case. In one embodiment, the invention may also feature an elastic band for securing the pad 12 on, around, or to a chair or other type of seat.

The pad 12 of the FIR heat emitting device 10 can conveniently be laid or worn over or wrapped around a body part of the user to relieve pain such as, for example, lower back pain. The below described preferred embodiments illustrate adaptation of this device and methods for using the device. Nonetheless, from the description of these embodiments, other aspects of the invention can be made and/or practiced based on the description provided below.

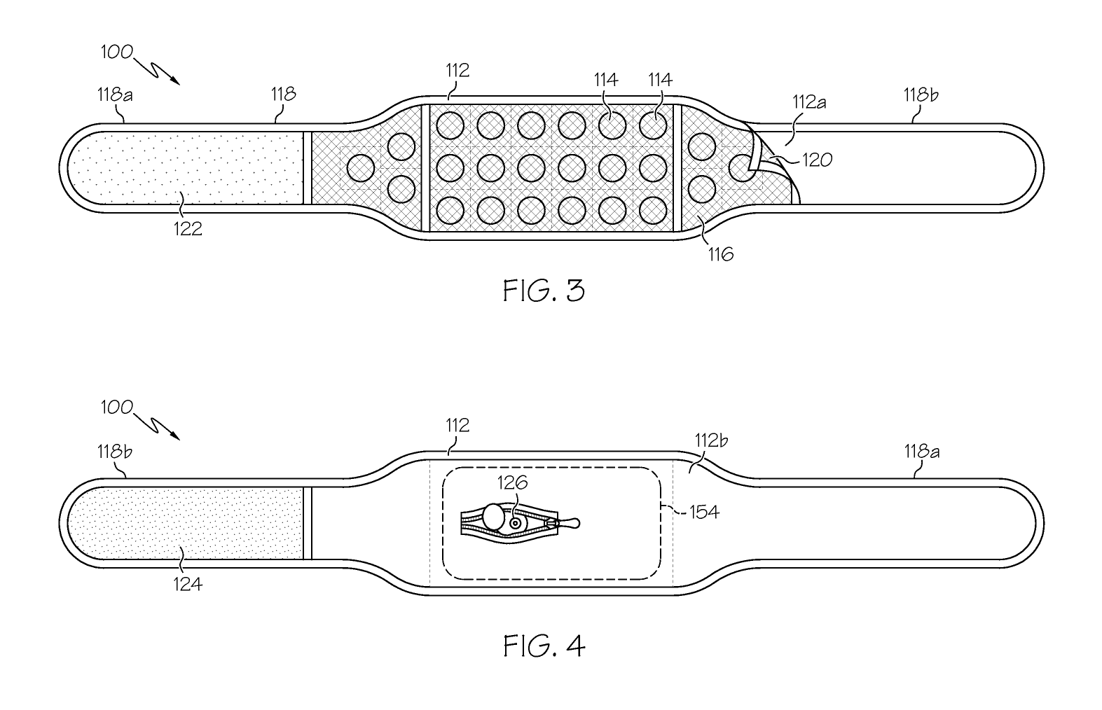

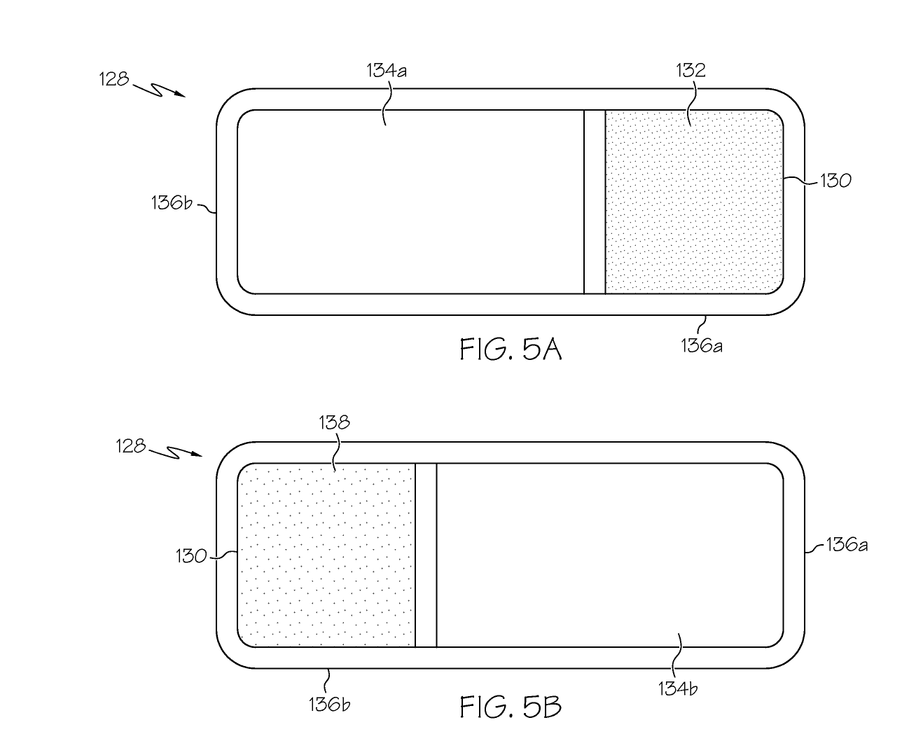

Referring now to FIGS. 3 and 4, an exemplary strap embodiment 100 of the invention is shown. The strap 100 can be used to secure the device 100 around a body part of a user such as, for example, a waist, an arm, a leg, a neck, a hand, a foot, a torso, or a back of the user. In an exemplary embodiment, the strap 100 is secured around the waist of a user. The strap 100 can include a pad 112, a plurality of far infrared (FIR) heat conducting elements 114, a device 116 for holding the FIR heat conducting elements 114 in place, and a fastener 118 that forms a strap for securing the pad 112 to a person. The pad 112 can be constructed in a manner and from materials similar to the pad 12 described above and can feature a front sheet of material 112a and a rear sheet of material 112b. The pad 112 may also be constructed from materials that are similar or identical to the materials for the pad 12 described above.

The device 116 can be similar to or the same as the mesh material 38 or other materials described herein and used to retain and fix the position of the plurality of FIR heating conducting elements 14 in the pad 12. The device 116 can include the front sheet of material 112a and a mesh material 120 attached thereto as shown in FIG. 3. The mesh material 120 can include apertures that allow the FIR heat conducting elements 114 (e.g., jade discs) to be seen and to allow far infrared heat emitted by the elements 114 to pass through to be absorbed by the user's skin.

The fastener 118 can include first and second straps 118a and 118b integrally attached to and forming a part of the pad 112. One of the straps (e.g., first strap 118a) may include hook fasteners 122 and the other strap (e.g., second strap 118b) may include loop fasteners 124 for connecting to the hook fasteners 122. In one embodiment, to accommodate most adults (e.g., having a waist size of about 28-38 inches), the strap can be approximately 46 inches long by 6 inches wide by 2 inches thick. In other embodiments, the strap can be about 28 to about 52 inches long, about 4 inches to about 12 inches wide, and about 0.5 inches to about 3 inches thick.

In an exemplary embodiment, the strap 100 can include a charging inlet 126 to which a power cord may be connected. In one embodiment, the power cord may be connected to the charging inlet 126 to charge a battery. The battery can serve as the strap's power source. In another embodiment, the power cord may be connected to the charging inlet 126 to provide a direct connection to a power source such as an electrical outlet of an electrical system or grid. The charging inlet 126 may be positioned on the rear sheet of material 112b and may be hidden within a pocket. The pocket may feature a zipper, buttons, snap fasteners, or other fastening means for closing the pocket in order to conceal the charging inlet 126.

The strap 100 can further include a remote control that may include features such as, for example, temperature control, a timer, and a power switch to turn the device off and on. The remote control may be connected to the strap 100 via a wired or a wireless connection.

The pad 112 and plurality of FIR heat conducting elements 114 can be constructed from the same materials and in the same or a similar manner as described above for the construction of the pad 12 and the plurality of FIR heat conducting elements 14. The plurality of FIR heat conducting elements 114 may be held in position and attached to the strap 100 by any suitable method such as sewing into individual pockets or cells, a mechanical or adhesive film fastener, or a hook-and-loop type fastener.

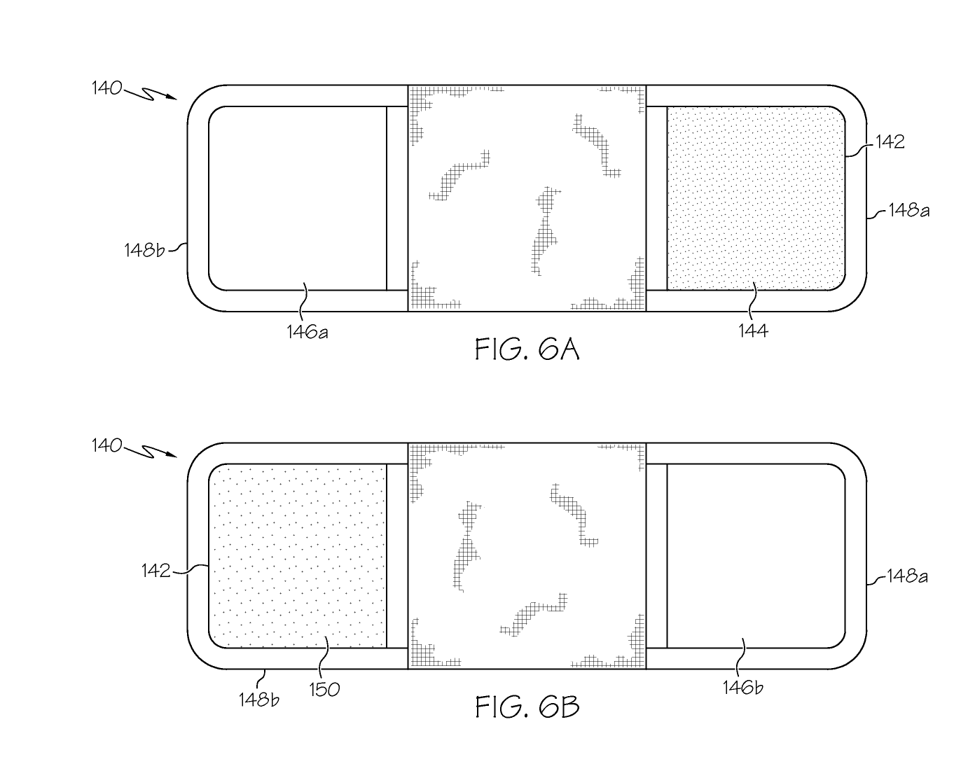

Referring now to FIGS. 5A and 5B, the strap 100 can include a short strap extension 128. The short strap extension 128 can be used to secure the strap 100 around the waist of a user who has a large waist size, e.g., a waist size of 36 to 48 inches. The short strap extension 128 may include fasteners 130 such as, for example, hook fasteners or loop fasteners, to connect to the fasteners 122 or 124 of one or both of the straps 118a and 118b. The short strap extension 128 can include hook fasteners 132 on a first side 134a at a first end 136a of the short strap extension. The short strap extension 128 can include loop fasteners 138 on a second side 134b at a second end 136b of the short strap extension. Thus, the hook fasteners 132 of the short strap extension 128 can be located on a reverse side and at an opposite end of the short strap extension from the position of the loop fasteners 138. In one embodiment, the short strap extension 128 may be constructed at least in part from an elastic material.

The hook fasteners 132 and the loop fasteners 138 of the short strap extension 128 can be connected to the corresponding hook fasteners 122 or loop fasteners 124 of the strap 100 to extend the length of the strap. Hook fasteners are used to connect to loop fasteners. For example, the hook fasteners 132 of the short strap extension 128 can be connected to the loop fasteners 124 of the strap's second strap 118b, and the loop fasteners 138 of the short strap extension 128 can connected to the hook fasteners 122 of the strap's first strap 118a.

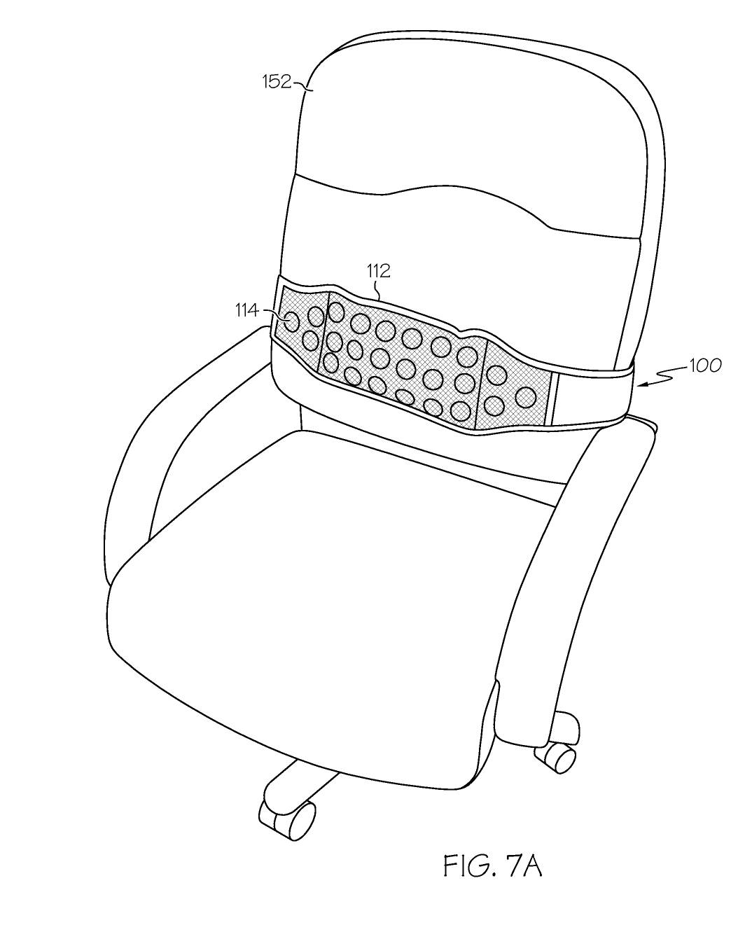

Referring now to FIGS. 6A and 6B, the strap 100 may also include a long strap extension 140 having features similar to those of the short strap extension 128. The long strap extension 140 can be used to secure the strap 100 around the waist of a user having a waist size of greater than 48 inches. The long strap extension 140 may include fasteners 142 such as, for example, hook fasteners or loop fasteners, to connect to the fasteners 122 or 124 of one or both of the straps 118a and 118b. The long strap extension 140 can include hook fasteners 144 on a first side 146a at a first end 148a of the long strap extension. The long strap extension 140 can include loop fasteners 150 on a second side 146b at a second end 148b of the long strap extension. Thus, the hook fasteners 144 of the long strap extension 140 can be located on a reverse side and at an opposite end of the long strap extension from the position of the loop fasteners 150. In one embodiment, the long strap extension 140 may be constructed at least in part from an elastic material.

The fasteners 130 of the short strap extension 128 may be connected to corresponding fasteners 142 of the long strap extension 140 to extend the length of the strap 100 to an even greater degree.

The hook fasteners 144 and the loop fasteners 150 of the long strap extension 140 can be connected to the corresponding hook fasteners 122 or loop fasteners 124 of the strap 100 to extend the length of the strap. Hook fasteners are used to connect to loop fasteners. For example, the hook fasteners 144 of the long strap extension 140 can be connected to the loop fasteners 124 of the strap's second strap 118b, and the loop fasteners 150 of the long strap extension 140 can connected to the hook fasteners 122 of the strap's first strap 118a.

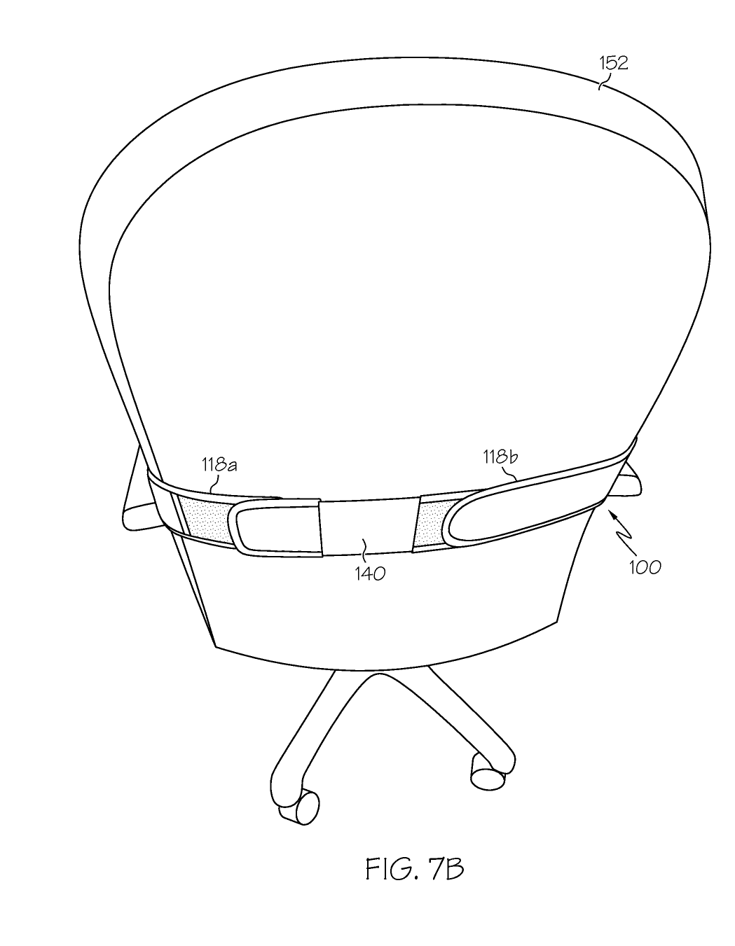

Referring now to FIGS. 7A and 7B, the long strap extension 140 might also be used to attach the strap around a chair 152. The strap 100 can oriented with the front sheet of material configured so that the FIR heat conducting elements 114 would be against the lower back of a user sitting in the chair. In another embodiment, the short strap extension 128 might also be used to attach the strap 100 around a chair 152. In still another embodiment, both the short strap extension 128 and the long strap extension 140 may be connected to the strap 100 and then to one another in order to attach the strap around a chair. The chair 152 can be a seat, a recliner, an office chair, a dining room chair, a straight-backed chair, or any other suitable chair around which the strap 100 may be attached.

Far infrared heat (having a wavelength equal to and including 5.6 to 1,000 micrometers (μm)) can be delivered to the user via the plurality of FIR heat conducting elements 14, 114. In the embodiment shown in FIG. 3, twenty-four circular convex jade discs each 1.5 inches in diameter and 0.3 ounces in weight are arranged in a regular pattern on the exterior surface of the front sheet of material (i.e., the side that abuts against a user when the strap 100 is worn). In other embodiments, a different number of FIR heat conducting elements may be attached to the pad 12 or to the strap 100 (e.g., 6, 7, 8, 9, 10, 12, 15, 20, 22, 25, 29, 30, 35, 40, 50, or 60 FIR heat conducting elements). The number and shape of the FIR heat conducting elements can be selected so that an appropriate amount of heat can be delivered to the user without the elements appearing uncomfortable, and allowing this invention to be uniquely soft and flexible.

In other embodiments, the composition, number, shape, and size of the FIR heat conducting elements can be varied. For example, although jade is preferred because of its ability to deliver far infrared heat in a manner preferred by users, other materials such as germanium, charcoal, composite stone, ceramic, and tourmaline ceramic may also be used. Jade's emission wavelength is equal to and includes 4 to 14 μm, which matches so closely to the human body's radiant energy that close to 93% of the infrared waves that reaches the user's skin is absorbed and is able to penetrate up to about 3 inches into tissue and permeate thoroughly the user's skin.

In an alternate embodiment, the FIR heat emitting device may be incorporated beneath an exterior surface of a piece of furniture. The exterior surface beneath which the FIR heat emitting device is incorporated can be a surface with which the user's body would normally come into contact. For example, the FIR heat emitting device can be installed beneath a seat and/or back surface of a chair, recliner, or sofa or beneath a top surface of a mattress.

In another embodiment, the pad may be incorporated on top of the exterior surface of the furniture piece so as to be capable of direct contact with the body of the user.

Another aspect of the invention includes the integration of the pad into furniture. The device may be integrated into the construction of furniture. The furniture may be used for human and/or animals. This integration could provide therapeutic benefits more fully discussed in this application to any individual or animal that uses the particular piece of furniture. Examples of such furniture include, but are not limited to, comfort chairs, recliners, office chairs, office task chairs, office executive chairs, sofas, couches, lift chairs, beds, pet chairs, and pet beds.

The invention, used in this aspect, may provide a far infrared heat emitting device integrated into the furniture, which device then may provide the therapeutic benefits of the device to reach the user of the furniture. When integrated into a comfort chair, recliner, lift chair, or sofa type furniture, the device provides a steady emission of far infrared heat for a user-determinable length of time through pad placements in the lumbar, back, and/or neck area(s) of the furniture and in the lower seat and leg positions. When similarly integrated into an office chair, task chair, executive chair, or similar furniture, the device may provide a steady emission of far infrared heat for a user-determinable length of time through pad placements in the lumbar and/or back area(s) of the office chairs. When integrated into a bed or pet furniture, the device may provide a steady emission of far infrared heat for a user-determinable length of time through pad placements in the area of the furniture that benefits the user the best. Such furniture-integrated devices may operate on battery power and/or electrical current.

According to an embodiment of the present invention, the device may include a carbon fiber matrix to create heat and/or far infrared frequencies. The carbon fiber matrix may be driven by the power source, which may convert the electrical power to infrared radiation.

### Methods of Use

The far infrared heat conducting device of the invention is generally intended to be used to provide far infrared heat to one or more body parts of a user such as, for example, the lower back, arm, leg, or neck of a person, to help relieve pain caused by inflammation or muscle soreness. To use the device, a human user places the pad (or pad of the strap) around or over a selected body part in a position where the plurality of far infrared heat conducting elements are positioned at the site of the user's pain (e.g., the lower back). The pad of the device is left in that position until the pain is relieved, e.g., about 0.2, 0.5, 1, 2, 3, 4, 5, 6, 7, 8 or more hours. Once the pain has diminished, the pad (or strap) may be removed by the user.

In another embodiment of the method, the user can be an animal (e.g., a pet) and a person can place the pad (or strap) over or around and in contact with a body part of the animal.

In another embodiment, a user employs the invention as discussed above however, the user need not be in pain or any discomfort. The pad may simply be used prophylactically as an ongoing health protocol.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A therapeutic device for positioning over or around a body part of a user, the device comprising:
a pad comprising a front surface and a rear surface;
a plurality of far infrared heat conducting elements;
a heating device;
a power source; and
a stone affixed to the pad by installation within a plurality of cells that are closed to prevent the stone from falling out or becoming dislodged.

2. The device of claim 1, wherein the stone further comprises jade, granite, tourmaline, or a combination thereof.

3. The device of claim 1, wherein the stone is a primer for creation of negative ions to be emitted.

4. The device of claim 1, further comprising shielding to at least partially insulate electromagnetic radiation created by other electronic devices.

5. The device of claim 1, wherein the pad comprises a strap to substantially secure the pad around the body part of the user.

6. The device of claim 5, wherein the strap is integrated with the pad as a single piece.

7. The device of claim 5, wherein the strap is extendable for securing the pad around a waist of the user having a waist size that is 36 inches or greater.

8. The device of claim 5, wherein the device is sized so as to be capable of attaching around a chair.

9. The device of claim 1, wherein the stone is at least partially crushed and integrated into the pad to create a negative ion environment.

10. The device of claim 1, wherein the power source comprises a power cord for connection to an outlet of an electrical system, a battery, or a combination thereof.

11. The device of claim 1, wherein the front surface of the pad comprises a pouch, wherein the pouch comprises a lining and wherein the lining comprises front and rear interior surfaces.

12. The device of claim 11, wherein the pad comprises individual cells sewn into the lining, wherein each of the plurality of far infrared heat conducting elements is positioned within one of the individual cells sewn into the lining.

13. The device of claim 12, wherein the lining comprises a mesh material with a plurality of apertures to permit unblocked emission of far infrared heat from the plurality of far infrared heat conducting elements.

14. The device of claim 1, wherein the heating device comprises an infrared frequency generating device.

15. The device of claim 14, wherein the infrared frequency generating device comprises a carbon-based material installed inside a pouch of the pad and constructed in a shape that provides close proximity to at least a majority of the plurality of far infrared heat conducting elements.

16. The device of claim 15, wherein an aura of negative ions is created by carbon-activated fiber passing a FIR frequency through the stone.

17. The device of claim 1, wherein the pad comprises a bioceramic infused FIR fabric.

18. The device of claim 1, wherein the pad comprises a distribution material to substantially uniformly distribute heat further, the distribution material comprising jade, granite, tourmaline, or a combination thereof.

19. The device of claim 1, wherein the plurality of far infrared heat conducting elements is connected to a mesh material, the front surface, or a combination thereof via a connecting means.

20. The device of claim 1, wherein the heating device comprises a carbon fiber matrix.

21. A therapeutic device for integrating into furniture, the device comprising:
a pad adjacent to the furniture;
a plurality of far infrared heat conducting elements;
a stone affixed to at least one surface of the pad via a fastener to distribute heat;
a heating device comprising a carbon fiber matrix; and
a power source.
